SEQUENCE LISTING (6 sequences)

SEQ ID NO: 1 - protein, 494 aa, Emergencia timonensis
  NON_STD 324: note = Wherein X at position 324 is defined as pyrrolysine
MGKFTHETDITGYNILSNEDLDALHESTLQLLEDYGLQMHGKEALDILAGAGCEVDYENDRVKFPRNLVNDAIEKCPAEFTLCGRDPAKDTVVGGKKVAYKNFGTGVFIFDPYTGELRESTKEDLGNVARFIDAIPEIDVFSIAVTAGDVNQKVRSLHEAEVVFNNLTKNFAHDLEGVKNTQRFIDMAAAIQGGYDKLRERPIVAMGACPNSPLEINENETSIIILSARHGLPIDILSMGLCGATTPATIAGTLVCTNAEILGGIVLAQIVNPGNKILYGSSTTIMDMKTAQSPVGAPEHAMCSAAVGQLGHYYGIPTNVGGTXADSKCFDVQLGHEKTMTNMLPALTGSNVIYGMGMMEMGMTMSYEQLLTDAEIVRMTRRILQGIPVNPDTMALDVIKAVGPANNYLAQKHTRKYMRQELSTTQYIDRTQRETWEKKGGMTMTEATKAKAIDILENYEVTPLPDDVKKRIHDIVVEGEEEAEELARFQAKKK

SEQ ID NO: 2 - protein, 484 aa, Emergencia timonensis
  NON_STD 323: note = Wherein X at position 323 is defined as pyrrolysine
MSFQFSKETRGYRLLTDQGIVTIHNATMDLMGQYGVRIFGTEAKKIYEEAGCDVDWESNMVKFPRELVEWAIEVTPGTFTMCGRHPKDDFVLGDGTVSFTSFGTGLQMFDLETGEVRDTDTEDLENFARFCDAIPEINAFTTAVAAQDAPGPLKDFYEAEAIFKNTTKHAILDAENGHNAKAIIDMGIAIAGSLQKLQERPFFTLCMCPNSPLEIHDGASQVIIETAKAGLPISILSMGLAGGTTPATLTGTFVVTNAEILAGIVLTQLIHEGNPVIYGSSTTVMDMRKATSPVGAPEHGMFGAMVAQMGKYYDIVTKVGGTXADSKVMDVQIGHEKTLTGLMPVLAGVDLVYGLGMIDMGMAISYEQFLMDAEFVRMFKRTEKEVMVDEDALALSVIKAVGPAGNYLSQRHTLAHMRDEISTTALIDRQMREKWQKDGAKDMPQRARELAKEILAKHRPEPLPADVQAELAAIIKREGDLLCR

SEQ ID NO: 3 - protein, 468 aa, Emergencia timonensis
  NON_STD 353: note = Wherein X at position 353 is defined as pyrrolysine
MGDQKKFFTRMGDGSPVYMTEEEIRADIKAGIDDAVLRGKIDPLSEEDTERIYEIVTMPGNIVGVQPGMEIVTSNDSGSDKISTKCGISTSRDTNALIHERVLGADSVDIGYSDYNYKTVKGVAKREATVLKQALTKATMPLFYGAMPNLGFYTKPDGPVDNWAMLLPEGKIKEAMAAQEEAVDHAVRDIVYVAEQMNDVGADAFMLDTSGAAGDADLLVSLKACEIIREKFPDMPVEIGMAGEFVLGMHGKLKYNGTRLAGLYPHKQVKVVEEAGASIFGAVVNTNCNKSFPWNIARVCTFIKACTDVAEIPVHANAGMGVCGIPMVENLPSDVVSRADKCLIEIGKVDGLXIGVGDPLGAEATHALAAGMGGIRTAGDLVMRLQLAKKMKINEAKAYVAEKLSVSVEELCDIVTMNDKREELGLGLAHVEPNAEANAGMEAKFRIQEVLDIKVNSVERFKERAGLK

SEQ ID NO: 4 - protein, 39 aa, Emergencia timonensis
  NON_STD 35: note = Wherein X at position 35 is defined as pyrrolysine
TAQSPVGAPEHAMCSAAVGQLGHYYGIPTNVGGTXADSK

SEQ ID NO: 5 - protein, 34 aa, Emergencia timonensis
ATMPLFYGAMPNLGFYTKPDGPVDNWAMLLPEGK

SEQ ID NO: 6 - DNA, 1480 bp, Emergencia timonensis
  source 1..1480: mol_type = genomic DNA
agagtttgatcctggctcaggatgaacgctggcggcgtgcctaacacatgcaagtcgagcgagaagccattgactgaaacttcggtagaaggatatggtggaaagcggcggacgggtgagtaacgcgtaggcaacctgccccttacagagggatagccattggaaacgatgattaaaacctcataacgcatcccccycacatggaggggawgccaaagattcatcggtaagggatgggcctgcgtctgattagcttgttggcggggtaacggcccaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagggatgaaggccttcgggtcgtaaacctctgtccttggggaagaaacaaatgacggtacccatggaggaagccccggctaactacgtgccagcagccgcggtaatacgtagggggcgagcgttatccggaattattgggcgtaaagagtgcgtaggtggttacctaagcgcagggtctaaggcaatggctcaaccattgttcgccctgcgaactgggctacttgagtgcaggagaggaaagcggaattcctagtgtagcggtgaaatgcgtagatattaggaggaacaccagtggcgaaggcggctttctggactgttactgacactgaggcacgaaagcgtggggagcaaacaggattagataccctggtagtccacgccgtaaacgatgagcactaggtgtcggggccgcaaggcttcggtgccgcagttaacgcattaagtgctccgcctggggagtacgcacgcaagtgtgaaactcaaaggaattgacggggacccgcacaagcagcggagcatgtggtttaattcgaagcaacgcgaagaaccttaccaggacttgacatcccactgacagatccctaaccggatccttcttcggacagtggagacaggtggtgcatggttgtcgtcagctcgtgtcgtgagatgttgggttaagtcccgcaacgagcgcaacccttgccattagttgccatcattcagttgggcactctaatgggactgccggggacaactcggaggaaggtggggatgacgtcaaatcatcatgccccttatgttctgggctacacacgtgctacaatggccggtacagcaagaagcaaaaccgcgaggtggagcaaatctcaaaaaccggtcccagttcggactgcaggctgaaacccgcctgcacgaagccggagttgctagtaatcgtggatcagaatgccacggtgaatgcgttcccgggtcttgtacacaccgcccgtcacaccatggaagttgggggtgcccgaagccggcagggaaatatgctgtctaaggcaaaaccaatgactggggtgaagtcgtaacaaggtagccgt

(19) United States
(12) Patent Application Publication
(10) Pub. No.: US 2024/0050492 A1
(43) Pub. Date: Feb. 15, 2024

(54) REDUCING TRIMETHYLAMINE OR TRIMETHYLAMINE-N OXIDE LEVELS IN A SUBJECT

(71) Applicant: Oregon State University, Corvallis, OR (US)

(72) Inventors: Veronika Kivenson, Corvallis, OR (US); Stephen J. Giovannoni, Corvallis, OR (US)

(73) Assignee: Oregon State University, Corvallis, OR (US)

(21) Appl. No.: 18/449,947

(22) Filed: Aug. 15, 2023

Related U.S. Application Data
(62) Division of application No. 17/511,141, filed on Oct. 26, 2021.
(60) Provisional application No. 63/105,720, filed on Oct. 26, 2020.

(51) Int. Cl. A61K 35/742; A61K 9/00; A23L 33/135; A23L 33/00; A23K 10/18

(52) U.S. Cl. CPC A61K 35/742; A61K 9/0053; A61K 9/0034; A23L 33/135; A23L 33/40; A23K 10/18; A23V 2002/00

(57) ABSTRACT

The present disclosure describes probiotic compositions comprising Emergencia timonensis and methods for using probiotic compositions comprising Emergencia timonensis to reducing TMA and/or TMAO levels in subjects in need thereof.

### CROSS-REFERENCE TO RELATED APPLICATION

This application is a divisional of U.S. application Ser. No. 17/511,141, filed Oct. 26, 2021, which claims the benefit of U.S. Provisional Application No. 63/105,720, filed Oct. 26, 2020, now expired, the disclosures of which are hereby incorporated by reference in their entirety.

### STATEMENT REGARDING SEQUENCE LISTING

The Sequence Listing XML associated with this application is provided in XML format and is hereby incorporated by reference into the specification. The name of the XML file containing the sequence listing is 3014-P18USDIV_Seq_List_20230811. The XML file is 14,025 bytes; was created on Aug. 11, 2023; and is being submitted via Patent Center with the filing of the specification.

### BACKGROUND

The human gut microbiome is increasingly recognized for its influential role in health. The gut microbiome has been implicated in cardiovascular disease (CVD), the leading cause of death in developed countries. Metabolic end products associated with the consumption of animal products have been shown to promote CVD; dietary sources of proatherogenic compounds include choline, phosphatidylcholine and carnitine. Gut microorganisms convert these compounds to trimethylamine (TMA), which in turn is converted to trimethylamine-N oxide (TMAO) through the action of host hepatic flavin monooxygenase. TMAO is a causative agent of CVD pathogenesis, therefore, elucidating pathways relevant to this compound is central to understanding human health.

The canonical view of CVD and TMAO involves the action of the gut microbiota in transforming precursor compounds from a range of animal-based dietary sources to TMA, which can then be converted to TMAO in the liver. While TMAO is frequently referred to as the sole breakdown product of TMA, a subset of methanogenic archaea originating from the gut have the ability to utilize TMA by an alternative pathway. This pathway is enabled by genetic code expansion (GCE), via insertion of the 22nd amino acid, pyrrolysine, in place of a TAG amber codon at conserved sites of the tri-, di-, and mono-methylamine methyltransferase genes, with methane as the end product. Due to the low abundance of these specialized archaea in the gut, their potential for reducing TMAO receives little attention. GCE-enabled TMA metabolism has also been described in some bacteria from environmental settings, including symbionts of gutless marine worms, as well as the Firmicutes bacterium, Acetohalobium arabaticum, isolated from a Crimean lagoon. Despite its potential importance, competition for TMA by GCE-enabled bacterial metabolism in the gut remains unexplored.

International Patent Application No. PCT/US2017/037869 discloses that Eubacterium strains can demethylate each quaternary amine (QA) that would otherwise form trimethylamine (TMA) and/or trimethylamine-N-oxide (TMAO). Therefore, Eubacterium limosum and members of Clostridial Clade XV, which are close relatives of E. limosum including E. aggregans, E. barkeri, and E. callanderi, can be taken as a probiotic supplement to treat or prevent against heart disease.

U.S. Pat. No. 10,238,695 discloses compositions containing a microorganism, preferably an Archaea, expressing a TMA methyltransferase and a TMA methyl group acceptor corrinoid protein, capable of metabolizing trimethylamine (TMA) in the presence of hydrogen in a human cavity, such as the intestine or the vagina, for use as a medicament for treating, reducing or eliminating TMA at the level of the human cavity. It further describes a composition containing a TMA methyltransferase and a TMA methyl group acceptor corrinoid protein. The described compositions are of use for treating trimethylaminuria, for treating vaginal fluids in the case of bacterial vaginosis and for reducing or eliminating odors due to TMA. These compositions are also of use for reducing the level of plasma TMAO, for preventing the formation of atheroma plaques and/or for preventing cardiovascular disease.

Koeth et al. demonstrates 1-carnitine accelerates atherosclerosis in mice via gut microbiota-dependent formation of trimethylamine (TMA) and trimethylamine N-oxide (TMAO) via a multistep pathway involving an atherogenic intermediate, γ-butyrobetaine (γBB). The contribution of γBB in gut microbiota-dependent 1-carnitine metabolism in humans is unknown. Extensive anaerobic subculturing of human feces identified no single commensal capable of 1-carnitine→TMA transformation, multiple community members that converted 1-carnitine to γBB, and only 1 Clostridiales bacterium, Emergencia timonensis, that converted γBB to TMA. In coculture, E. timonensis promoted the complete 1-carnitine→TMA transformation (Koeth R A, Lam-Galvez B R, Kirsop J, Wang Z, Levison B S, Gu X, Copeland M F, Bartlett D, Cody D B, Dai H J, Culley M K, Li X S, Fu X, Wu Y, Li L, DiDonato J A, Tang W H W, Garcia-Garcia J C, Hazen S L. 2019. L-Carnitine in omnivorous diets induces an atherogenic gut microbial pathway in humans (J. Clin. Invest. 129:373-387)).

For all these reasons, a need exists for pharmaceutical and/or probiotic agents and methods for reduction of TMA and TMAO levels in subjects in need thereof.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Emergencia timonensis is described in the literature as promoting the breakdown of carnitine from dietary sources to trimethylamine (TMA). The inventors of the present disclosure discovered that Emergencia timonensis is also actively utilizing TMA in the human gut and breaking it down further to dimethylamine, thus depleting the quantity of TMA available for host hepatic processes, and that the genome of Emergencia timonensis encodes unique proteins involved in this process. The utilization of TMA is achieved, at least in part, by the action of pyrrolysine-containing trimethylamine methyltransferase proteins. SEQ ID NO:1 and SEQ ID NO:2 are amino acid sequences for the pyrrolysine-containing trimethylamine methyltransferase proteins, which demethylates trimethylamine, resulting in removal of the methyl group from this compound, and production of the compound, dimethylamine. The SEQ ID NO:1 and SEQ ID NO:2 proteins are responsible for the key step in which trimethylamine is converted to dimethylamine.

Disclosed herein are embodiments of a probiotic composition and methods of using this probiotic composition to reduce TMA and trimethylamine N-oxide (TMAO) in subjects with a disease that is treatable by reducing elevated TMA and TMAO levels.

In one aspect, the disclosure provides a probiotic composition. In some embodiments, the probiotic composition can comprise Emergencia timonensis and a pharmaceutically acceptable carrier or excipient. In some embodiments, the Emergencia timonensis can be selected from a strain that is 99% identical to strain SN18. In some embodiments, the Emergencia timonensis can be selected from a strain that is 99% identical to strain AM07-24. In some embodiments, the Emergencia timonensis can be selected from a strain that is 99% identical to isolate MGYG-HGUT-01547. In still other embodiments, the Emergencia timonensis can be strain SN18. In some embodiments, the Emergencia timonensis can comprise a combination of at least two of strain SN18, strain AM07-24, isolate MGYG-HGUT-01547, and combinations thereof. In some embodiments, the Emergencia timonensis can express a protein having an amino acid sequence that is at least 90% identical to the amino acid sequence of SEQ ID NO:1 and/or amino acid sequence of SEQ ID NO:2.

In some embodiments, the probiotic composition can be formulated for a human or an animal. In some embodiments, the probiotic formulation can be compounded as a food or as a pharmaceutical preparation. In still other embodiments, the pharmaceutical preparation can be a capsule, a suppository, an enema, a powder, a foam, a gel, a suspension or a fecal transplant. In some embodiments, the pharmaceutical preparation can be a vaginal suppository. In some embodiments, the pharmaceutical preparation can be formulated to be released in the intestine of a subject following administration of the probiotic composition to the subject. In some embodiments, the pharmaceutical preparation can be formulated to be released in the urogenital tract of a subject following administration of the probiotic composition to the subject. In some embodiments, the probiotic composition can comprise from 1 to 100 billion, from 1 to 10 billion, from 1 to 5 billion, or from 10 million to 1 billion colony forming units. In some embodiments, Emergencia timonensis expresses at least one TMA utilizing protein under anaerobic growth conditions.

In another aspect, the disclosure provides a method for reducing or maintaining the amount of TMA or TMAO in a subject. In some embodiments, the method can comprise administering an effective amount of a probiotic composition. In some embodiments, the probiotic composition can comprise Emergencia timonensis and a pharmaceutically acceptable carrier or excipient. In some embodiments, the Emergencia timonensis can be selected from a strain that is 99% identical to strain SN18. In some embodiments, the Emergencia timonensis can be selected from a strain that is 99% identical to strain AM07-24. In some embodiments, the Emergencia timonensis can be selected from a strain that is 99% identical to isolate MGYG-HGUT-01547. In still other embodiments, the Emergencia timonensis can be strain SN18. In some embodiments, the Emergencia timonensis can comprise a combination of at least two of strain SN18, strain AM07-24, isolate MGYG-HGUT-01547, and combinations thereof. In some embodiments, the Emergencia timonensis can express a protein having an amino acid sequence that is at least 90% identical to the amino acid sequence of SEQ ID NO:1 and/or the amino acid sequence of SEQ ID NO:2.

In some embodiments, the probiotic composition can be formulated for a human or an animal. In some embodiments, the probiotic formulation can be compounded as a food or as a pharmaceutical preparation. In still other embodiments, the pharmaceutical preparation can be a capsule, a suppository, an enema, a powder, a foam, a gel, a suspension or a fecal transplant. In some embodiments, the pharmaceutical preparation can be a vaginal suppository. In some embodiments, the pharmaceutical preparation can be formulated to be released in the intestine of a subject following administration of the probiotic composition to the subject. In some embodiments, the pharmaceutical preparation can be formulated to be released in the urogenital tract of a subject following administration of the probiotic composition to the subject. In some embodiments, the probiotic composition can comprise from 1 to 100 billion, from 1 to 10 billion, from 1 to 5 billion, or from 10 million to 1 billion colony forming units. In some embodiments, Emergencia timonensis expresses at least one TMA utilizing protein under anaerobic growth conditions.

In some embodiments, the effective amount of the probiotic composition can be an amount that reduces TMA or TMAO at or below a level of TMA or TMAO found in a healthy subject. In some embodiments, the subject can have or can be suspected of having, for example, cardiovascular disease, thrombosis, trimethylaminuria, bacterial vaginosis, or a combination thereof. In some embodiments, the subject can have an elevated TMA level or an elevated TMAO level. In some embodiments, the TMA level and/or the TMAO level can be determined by measuring the elevated TMA level and/or the elevated TMAO level in the urine. The level of TMA and/or TMAO can be measured, for example, by using liquid chromatography-mass spectrometry.

In another aspect, the disclosure provides a method for treating a condition treatable by reducing or maintaining the level of TMA in the gastrointestinal tract and/or the urogenital tract of a subject. In some embodiments, the method can comprise administering an effective amount of a probiotic composition to the gastrointestinal tract or the urogenital tract of the subject in need thereof. In some embodiments, the probiotic composition can comprise Emergencia timonensis and a pharmaceutically acceptable carrier or excipient. In some embodiments, the Emergencia timonensis can be selected from a strain that is 99% identical to strain SN18. In some embodiments, the Emergencia timonensis can be selected from a strain that is 99% identical to strain AM07-24. In some embodiments, the Emergencia timonensis can be selected from a strain that is 99% identical to isolate

MGYG-HGUT-01547. In still other embodiments, the Emergencia timonensis can be strain SN18. In some embodiments, the Emergencia timonensis can comprise a combination of at least two of strain SN18, strain AM07-24, isolate MGYG-HGUT-01547, and combinations thereof. In some embodiments, the Emergencia timonensis can express a protein having an amino acid sequence that is at least 90% identical to the amino acid sequence of SEQ ID NO:1 and/or to the amino acid sequence of SEQ ID NO:2.

In some embodiments, the probiotic composition can be formulated for a human or an animal. In some embodiments, the probiotic formulation can be compounded as a food or as a pharmaceutical preparation. In still other embodiments, the pharmaceutical preparation can be a capsule, a suppository, an enema, a powder, a foam, a gel, a suspension or a fecal transplant. In some embodiments, the pharmaceutical preparation can be a vaginal suppository. In some embodiments, the pharmaceutical preparation can be formulated to be released in the intestine of a subject following administration of the probiotic composition to the subject. In some embodiments, the pharmaceutical preparation can be formulated to be released in the urogenital tract of a subject following administration of the probiotic composition to the subject. In some embodiments, the probiotic composition can comprise from 1 to 100 billion, from 1 to 10 billion, from 1 to 5 billion, or from 10 million to 1 billion colony forming units. In some embodiments, Emergencia timonensis expresses at least one TMA utilizing protein under anaerobic growth conditions.

In some embodiments, the effective amount of the probiotic composition can be an amount that reduces TMA and/or TMAO at or below a level of TMA and/or TMAO found in a healthy subject. In some embodiments, the subject can have or can be suspected of having cardiovascular disease, thrombosis, trimethylaminuria, bacterial vaginosis, or a combination thereof. In some embodiments, the subject can have an elevated TMA level and/or an elevated TMAO level. In some embodiments, the TMA level and/or the TMAO level can be determined by measuring the elevated TMA level and/or the elevated TMAO level in the urine. The level of TMA and/or TMAO can be measured, for example, by using liquid chromatography-mass spectrometry.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic overview of the eight steps leading to the discovery of the bacteria of the invention. In Step 1, a study was undertaken to explore and survey marine microbial ecology; Step 2 included researching publicly available sequencing data; Step 3 identified specialized bacterial genomes from Steps 1 and 2; in Step 4, a custom translation table was created; in Step 5, the data collected in Steps 1-3 were reexamined using the custom DNA translation table; in Step 6, specialized gut bacteria were identified; in Step 7, gene expression for the specialized gut bacteria identified in Step 6 was determined; and in Step 8, sequences of the expressed genes were identified.

FIGS. 2A through 2B is an overview of the invention. FIG. 2A. TMA is converted to TMAO in the liver via a host hepatic flavin monooxygenase 3 (FMO-3), leading to increased risk for cardiovascular disease. FIG. 2B. A new view of TMA metabolism proposed in this disclosure; the specialized gut bacteria, Emergencia timonensis, increase in abundance and use genetic code expansion to augment metabolism, thereby reducing the amount of TMA available for conversion to TMAO.

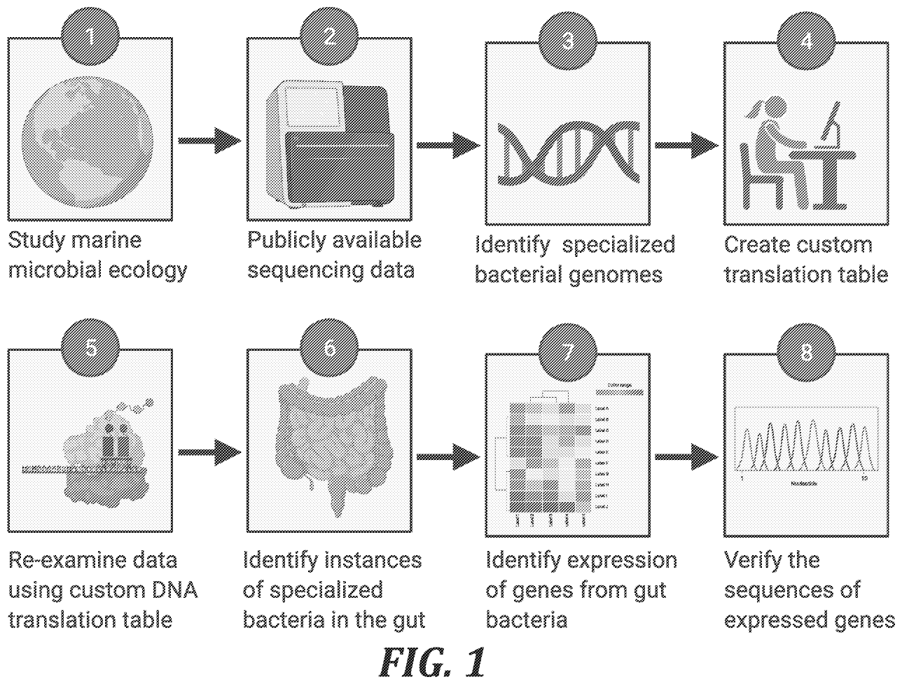

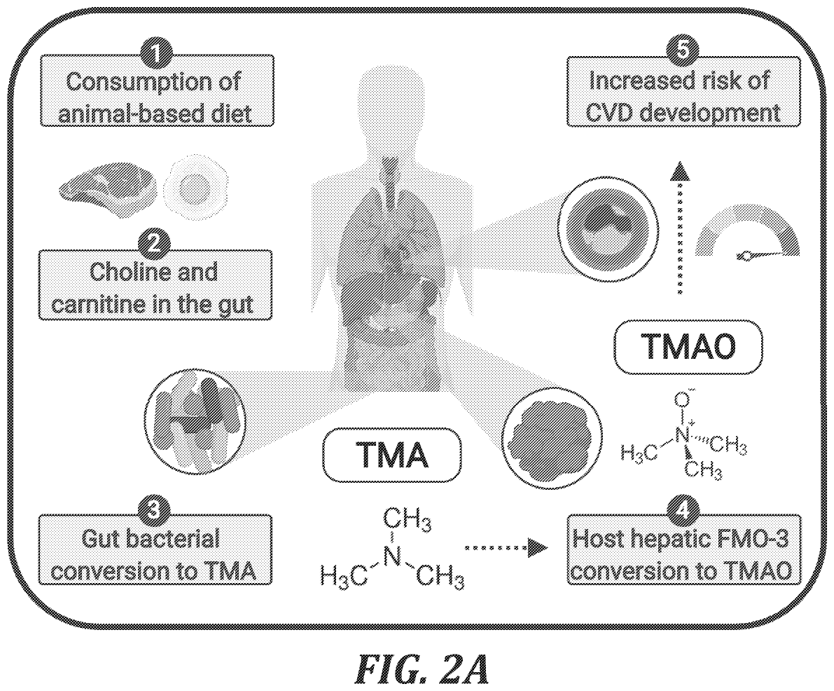

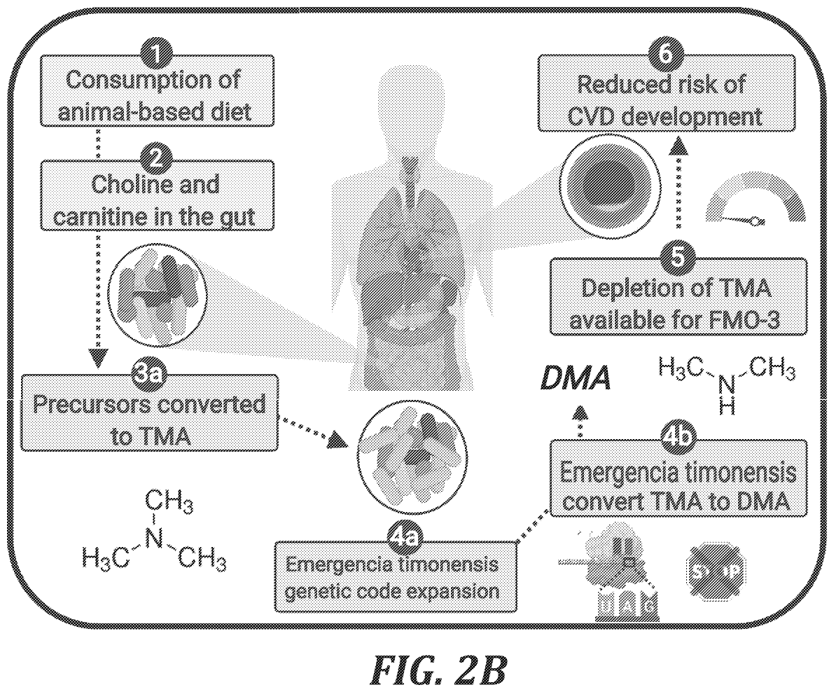

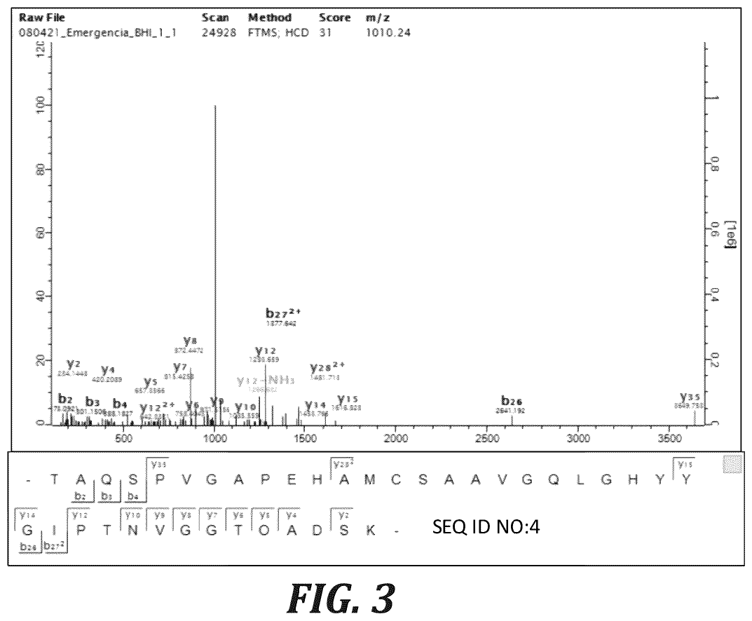

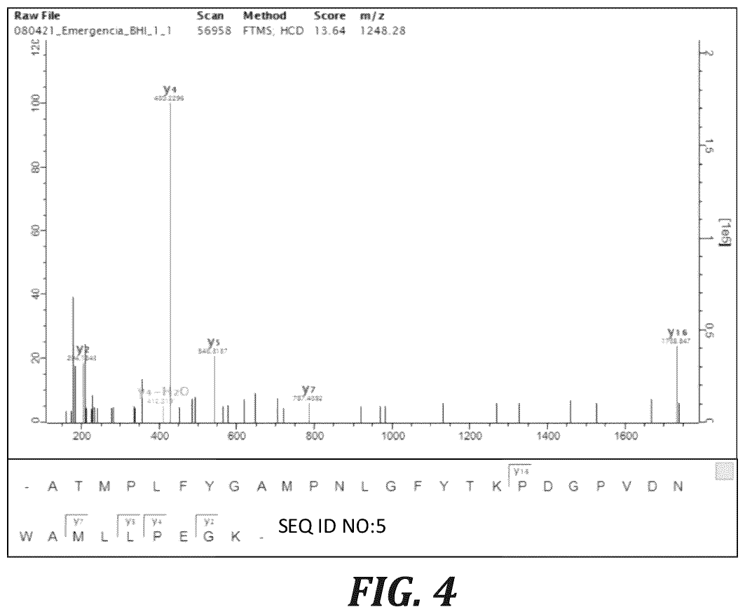

### DETAILED DESCRIPTION

The present disclosure relates to the use of microorganisms capable of limiting, stabilizing, or reducing the amount of trimethylamine (TMA) in a human or animal subject. TMA metabolism can occur in any anaerobic space in a subject's body, such as the gut. In embodiments, this disclosure describes using microorganisms in the intestines, vaginal cavity, or other anaerobic space of a subject to reduce TMA levels in the intestine, vaginal cavity, or other anaerobic space. Anaerobic areas within human subjects are where TMA metabolism can take place. Currently, the evidence of this can be seen in the samples originating from the human gut.

In embodiments, the microorganisms are bacterium from the genus Emergencia, for example, the bacterium is Emergencia timonensis.

In some embodiments, the bacterium is Emergencia timonensis strain or isolate identified by the accessions as follows:
NZ_FLKM00000000 for Emergencia timonensis strain SN18;
NZ_QRMS00000000 for Emergencia timonensis strain AM07-24; or
NZ_CABKWE000000000 for Emergencia timonensis isolate MGYG-HGUT-01547.

In some embodiments, the Emergencia timonensis comprises a gene encoding a protein of SEQ ID NO:1 and/or SEQ ID NO:2. In some embodiments, the Emergencia timonensis expresses a protein of SEQ ID NO:1 and/or SEQ ID NO:2 or a functional variant thereof. This SEQ ID NO:1 and SEQ ID NO:2 are amino acid sequences for the pyrrolysine-containing trimethylamine methyltransferase protein, which demethylates trimethylamine, resulting in removal of the methyl group from this compound, and production of the compound, dimethylamine. When L-carnitine, choline, and other compounds are introduced to the gut microbiome from dietary sources, they are converted to trimethylamine. The SEQ ID NO:1 and SEQ ID NO:2 proteins are responsible for the key step in which trimethylamine is converted to dimethylamine

The inventors of this disclosure unexpectedly discovered that Emergencia timonensis expresses a TMA utilizing metabolic pathway. Prior to this surprising discovery, Emergencia timonensis was reported to be a maker of TMA, specifically by the conversion of γBB to TMA as reported by Koeth et al. The inventors further discovered that not only does Emergencia timonensis express the TMA utilizing metabolic pathway, but that it does so when it is present in the human gut as part of the gut microbiome. The unexpected and surprising discoveries made by the inventors allow for the formulation of compositions and methods of using Emergencia timonensis for limiting, maintaining, or reducing the amount of TMA present in a subject.

In one aspect, the disclosure provides a probiotic composition. In some embodiments, the probiotic composition can comprise Emergencia timonensis and a pharmaceutically acceptable carrier or excipient.

In some embodiments, any suitable pharmaceutically acceptable carrier or excipients can be used in the claimed probiotic composition. In some embodiments, the pharmaceutically acceptable carrier or excipient can include one or more agents that stimulate activity of Emergencia timonensis and/or preserve viability thereof, including carnitine or TMA.

In some embodiments, the probiotic composition can be used as a regularly consumed probiotic to promote cardiovascular health or as a method for suppressing levels of TMA in patients where TMA levels are too high, requiring intervention.

As used herein, the term “pharmaceutically acceptable” can refer to a component, e.g., carrier or excipient, that is not biologically or otherwise undesirable, i.e., the component can be incorporated into a pharmaceutical formulation of the invention and administered to a subject as described herein without causing any significant undesirable biological effects or interacting in a deleterious manner with any of the other components of the formulation in which it is contained.

The term “excipient” can include any and all solvents, diluents, or other liquid vehicles, dispersion or suspension aids, surface active agents, isotonic agents, thickening or emulsifying agents, preservatives, solid binders, lubricants and the like, as suited to the particular dosage form desired. The term “carrier” as used herein refers to any substrate used in the process of probiotic delivery, which can improve the stability and/or effectiveness of the probiotic. In some embodiments, specific examples can include water, physiologically acceptable buffered saline solutions such as phosphate buffered saline solutions pH 7.0 to 8.0. In still other embodiments, suitable pharmaceutical carriers can include sterile water, salt solutions (such as Ringer's solution), alcohols, polyethylene glycols, gelatin, carbohydrates such as lactose, amylose or starch, magnesium stearate, talc, silicic acid, viscous paraffin, fatty acid esters, hydroxymethylcellulose, polyvinylpyrrolidone, and the like.

In some embodiments, the Emergencia timonensis can be selected from a strain that is 99% identical to strain SN18. In some embodiments, the Emergencia timonensis can be selected from a strain that is 99% identical to strain AM07-24. In some embodiments, the Emergencia timonensis can be selected from a strain that is 99% identical to isolate MGYG-HGUT-01547. In still other embodiments, the Emergencia timonensis can be strain SN18. In some embodiments, the Emergencia timonensis can comprise a combination of at least two of strain SN18, strain AM07-24, isolate MGYG-HGUT-01547, and combinations thereof. As used herein, percent identity refers to a comparison of the nucleotide sequence of the 16s rRNA gene sequence in, for example, strain SN18, e.g., the Emergencia timonensis can be selected from a strain that is 99% identical to the nucleotide sequence of the 16s rRNA gene sequence in strain SN18. The 16s rRNA gene sequence accession number for strain SN18 is LN998061. The 16s rRNA gene sequences for strain AM07-24 and isolate MGYG-HGUT-01547 were taken from their genomic sequences. The genomic sequence accession number for strain AM07-24 is GCA_003472745. The genomic sequence accession number for isolate MGYG-HGUT-01547 is GCA_902375955.

In some embodiments, the Emergencia timonensis can express a protein having an amino acid sequence that is at least 90% identical to the amino acid sequence of SEQ ID NO:1 and/or at least 90% identical to the amino acid sequence of SEQ ID NO:2.

The term “strain” as used herein refers to a genetic variation or subtype of a microorganism, e.g., SB18 is a strain of Emergencia timonensis. Methods for bacteria strain typing can include any standard methods well known to one of ordinary skill in the art.

In some embodiments, the probiotic composition can be formulated for a human or an animal. In some embodiments, the probiotic formulation can be compounded as a food or as a pharmaceutical preparation.

In some embodiments, the food can be any substance consumed to provide nutritional support for a human or an animal. In some embodiments, a probiotic composition can be formulated into dietary supplements and/or fermented dairy products. In some embodiments, the probiotic composition can be formulated into a food according to standard methods well known to one of ordinary skill in the art. In some embodiments, food can be a supplement such as a probiotic supplement. In some embodiments, the probiotic composition can be formulated into a probiotic supplement according to standard methods well known to one of ordinary skill in the art.

In some embodiments, the probiotic composition can be administered to a subject or self-administered by a subject. In some embodiments, a probiotic composition can be administered by ingesting an edible formulation. Examples of edible formulations include dietary supplements, probiotic capsules, probiotics food and dairy products, and other formulations suitable for ingestion.

As used herein, the terms “individual,” “host,” “subject,” and “patient” are used interchangeably. These terms typically refer to a human or an animal. Animals include, but not limited to, primates, including simians, equines (e.g., horses), canines (e.g., dogs), felines, various domesticated livestock (e.g., ungulates, such as swine, pigs, goats, sheep, and the like), as well as domesticated pets and animals maintained in zoos.

In still other embodiments, the pharmaceutical preparation can be encapsulated in a capsule for taking or administering as a probiotic. In some embodiments, the pharmaceutical preparation can include a suppository, an enema, a powder, a foam, a gel, a suspension or a fecal transplant. In some embodiments, the pharmaceutical preparation can be a vaginal suppository. In some embodiments, the pharmaceutical preparation can be lyophilized or freeze dried. In some embodiments, the pharmaceutical preparation can be in the form of a spray-dried powder. In some embodiments, the pharmaceutical preparation can be formulated into one of the preparations described above, e.g., capsule, suppository, powder, and the like, according to standard methods well known to one of ordinary skill in the art.

In some embodiments, the pharmaceutical preparation can be mixed with auxiliary agents, e.g., lubricants, stabilizers, wetting agents, emulsifiers, salts for influencing osmotic pressure, buffers, coloring, and/or aromatic substances and the like which do not deleteriously react with the active compounds.

A condition related to excess levels of TMA is bacterial vaginosis. In some embodiments, the present disclosure provides for vaginal suppositories that are useful for limiting, stabilizing, or reducing the amount of TMA in a subject's vaginal cavity.

In some embodiments, a probiotic composition can be administered by inserting a rectal suppository. In some embodiments, fecal microbiota transfer (FMT) can be used wherein subjects receive FMT from individuals with Emergencia timonensis in their fecal samples.

In some embodiments, the pharmaceutical preparation can be formulated to be released in the intestine of a subject following administration of the probiotic composition to the subject. In some embodiments, the pharmaceutical preparation can be formulated to be released in the urogenital tract of a subject following administration of the probiotic composition to the subject. In some embodiments, the pharmaceutical preparation is formulated to be released in the body cavity to which it is being delivered, e.g., in the intestine or the urogenital tract of a subject following administration of the probiotic composition.

As used herein, the term “body cavity” refers to a cavity or lumen of a particular organ. For example, body cavity can include, but is not limited to, a patient's vaginal cavity and/or a patient's gastrointestinal cavity. In some embodiments, the methods of the disclosure described above can be performed by delivering the compositions of the disclosure to a patient's body cavity. In some embodiments, the body cavity can be part of the gastrointestinal tract or the urogenital tract.

As used herein, the term “release” or “released” refers to delivery of the probiotic composition to a specific location within the gastrointestinal tract, e.g., duodenum, or the urogenital tract. In some embodiments, the probiotic composition is loaded into a capsule, wherein the capsule has an enteric coating. In some embodiments, the enteric coating is a reverse enteric coating. In some embodiments, the enteric coating solubilizes in a pH that matches its targeted location. Methods of formulating enteric coating to solubilize at certain locations along gastrointestinal tract or the urogenital tract can include any standard methods well known to one of ordinary skill in the art.

As used herein, the term “controlled-release” or “controlled-release drug delivery” or “extended release” refers to release or administration of a certain dosage of the probiotic composition in a controlled manner in order to achieve the desired pharmacokinetic profile in vivo based on a certain absorption, distribution, metabolism, and excretion of the probiotic composition. An aspect of “controlled” drug delivery is the ability to manipulate the formulation and/or dosage form in order to establish the desired kinetics of drug release.

In some embodiments, the probiotic composition can comprise from 1 to 100 billion, from 1 to 10 billion, from 1 to 5 billion, or from 10 million to 1 billion colony forming units. In some embodiment, the probiotic composition comprises sufficient number of Emergencia timonensis to establish a colony of Emergencia timonensis in the cavity to which it has been delivered.

In some embodiments, Emergencia timonensis comprises a gene encoding a protein of SEQ ID NO:1 and/or SEQ ID NO:2. In some embodiments, Emergencia timonensis expresses a protein of SEQ ID NO:1 and/or SEQ ID NO:2 or a functional variant thereof. As used herein, the term “expressing” or “producing” are used interchangeable to refer to proteins that are made by Emergencia timonensis, wherein the proteins are made under anerobic conditions as explained in Example 3.

In some embodiments, SEQ ID NO:1 and SEQ ID NO:2 are amino acid sequences for the pyrrolysine-containing trimethylamine methyltransferase proteins, which demethylate TMA, resulting in removal of the methyl group from this compound, and production of the compound, dimethylamine (SEQ ID NO:3), i.e., reducing TMA. As used herein, this process whereby pyrrolysine-containing trimethylamine methyltransferase proteins demethylate TMA, to produce dimethylamine is one embodiment describing how these methyltransferase proteins “utilize” TMA and by utilizing TMA, these proteins reduce TMA levels and consequently reduce TMAO levels. In some embodiments, reducing TMA has the effect of reducing TMAO because reducing TMA decreases the pool of TMA available for the liver, which converts TMA into TMAO.

In some embodiments, Emergencia timonensis can produce TMA utilizing proteins under anaerobic growth conditions as detailed in Example 3. In some embodiments, the anaerobic growth conditions are created with an anaerobic chamber. The anaerobic conditions can be any standard anaerobic condition well known to one of ordinary skill in the art. In some embodiments, the anaerobic chamber can be chosen from any standard anaerobic chamber well known to one of ordinary skill in the art.

In another aspect, the disclosure provides a method for limiting, maintaining, or reducing the amount of TMA or TMAO in a subject. In some embodiments, the method is useful for limiting, maintaining, or reducing the amount of TMA or TMAO in a subject at risk for a disease or condition treatable by reduction of TMA or TMAO. In some embodiments, the method is useful for limiting, maintaining, or reducing the amount of TMA or TMAO in a subject suffering from a disease or condition treatable by reduction of TMA or TMAO. As used herein, reducing the level of TMA and/or TMAO means lowering TMA and/or TMAO from an elevated level to a level equivalent to a healthy subject. As used herein, limiting or maintaining the level of TMA and/or TMAO means preventing TMA and/or TMAO levels from increasing in a healthy subject. In some embodiments, the method can be used to prevent a condition or disease associated with increased TMA and/or TMAO levels.

In some embodiments, the method can comprise administering an effective amount of a probiotic composition. In some embodiments, the probiotic composition can comprise Emergencia timonensis and a pharmaceutically acceptable carrier or excipient. In some embodiments, the Emergencia timonensis can be selected from a strain that is 99% identical to strain SN18. In some embodiments, the Emergencia timonensis can be selected from a strain that is 99% identical to strain AM07-24. In some embodiments, the Emergencia timonensis can be selected from a strain that is 99% identical to isolate MGYG-HGUT-01547. In still other embodiments, the Emergencia timonensis can be strain SN18. In some embodiments, the Emergencia timonensis can comprise a combination of at least two of strain SN18, strain AM07-24, isolate MGYG-HGUT-01547, and combinations thereof. In some embodiments, the Emergencia timonensis can express a protein having an amino acid sequence that is at least 90% identical to SEQ ID NO:1 or SEQ ID NO:2.

In some embodiments, the probiotic composition can be formulated for a human or an animal. In some embodiments, the probiotic formulation can be compounded as a food or as a pharmaceutical preparation. In still other embodiments, the pharmaceutical preparation can be a capsule, a suppository, an enema, a powder, a foam, a gel, a suspension or a fecal transplant. In some embodiments, the pharmaceutical preparation can be a vaginal suppository. In some embodiments, the pharmaceutical preparation can be formulated to be released in the intestine of a subject following administration of the probiotic composition to the subject. In some embodiments, the pharmaceutical preparation can be formulated to be released in the urogenital tract of a subject following administration of the probiotic composition to the subject. In some embodiments, the probiotic composition can comprise from 1 to 100 billion, from 1 to 10 billion, from 1 to 5 billion, or from 10 million to 1 billion colony forming units. In some embodiment, the probiotic composition comprises sufficient number of Emergencia timonensis to establish a colony of Emergencia timonensis in the cavity to which it has been delivered.

In some embodiments, the effective amount of the probiotic composition can be an amount that reduces TMA or TMAO at or below a level of TMA or TMAO found in a healthy subject population, i.e., population not suffering from the disease or condition treatable by reduction of the amount of TMA or TMAO. In some embodiments, the range of urinary TMA levels in healthy subjects are ranges between 0.70 μM and 4.39 μM. In some embodiments, reducing the amount of TMA by the methods of the disclosure results in a reduction in the amount of TMAO in the patient.

In some embodiments, the subject can have or can be suspected of having cardiovascular disease, thrombosis, trimethylaminuria, bacterial vaginosis, or a combination thereof. In some embodiments, the subject can have an elevated TMA level or an elevated TMAO level. In some embodiments, the TMA level and the TMAO level can be determined by measuring the elevated TMA level and the elevated TMAO level in the urine using liquid chromatography-mass spectrometry (LC/MS/MS).

In some embodiments, LC/MS/MS quantitation of TMA and TMAO in urine can be performed, and plasma/serum and urine TMAO can be simultaneously measured with other TMA-related metabolites such as choline, carnitine, and γ-butyrobetaine by LC/MS/MS after precipitation of protein with methanol. There is a wider range in concentration in urinary TMAO as compared to TMA as measured in healthy humans. The normal range of urinary TMA levels in healthy subjects are between 0.70 and 4.39 μM. For TMAO, the range is 52.0 to 1141.0 μM. Normal Ranges are reported in the paper entitled, “Simultaneous Measurement of Urinary Trimethylamine (TMA) and Trimethylamine N-Oxide (TMAO) by Liquid Chromatography-Mass Spectrometry,” Molecules, April 17; 25(8):1862 (2020), which is incorporated herein by this reference.

In another aspect, the disclosure provides a method for treating a condition treatable by limiting, maintaining, or reducing the level of TMA in the gastrointestinal tract or the urogenital tract of a subject. The various compositions described in this disclosure can be administered by or to subjects who either exhibit or are at risk of developing conditions related to undesirable levels of TMA. Examples of such conditions include, but are not limited to, cardiovascular disease, bacterial vaginosis, or trimethylaminuria.

In some embodiments, the methods of the disclosure can be performed by delivering the compositions of the disclosure to a body cavity of the patient. In some embodiments, the body cavity is a part of gastrointestinal tract or urogenital tract.

Trimethylaminuria is a metabolic disorder. Subjects afflicted with trimethylaminuria fail to convert TMA into a compound called trimethylamine N-oxide. As a result, TMA builds up in the body of the subject. Currently, there is no cure for trimethylaminuria, and subjects who suffer from it often resort to avoiding foods that contribute to trimethylamine build up. Trimethylaminuria is commonly diagnosed using a urine test and looking for higher than normal levels of TMA. In some embodiments, trimethylaminuria can be treated by administration of the disclosed probiotic composition formulations through fecal microbiota transfer (FMT) from individuals with Emergencia timonensis in their fecal microbiome.

As used herein, the terms “cardiovascular disease” (CVD) or “cardiovascular disorder” are terms used to classify numerous conditions affecting the heart, heart valves, and vasculature (e.g., arteries and veins) of the body and encompasses diseases and conditions including, but not limited to arteriosclerosis, atherosclerosis, myocardial infarction, acute coronary syndrome, angina, congestive heart failure, aortic aneurysm, aortic dissection, iliac or femoral aneurysm, pulmonary embolism, primary hypertension, atrial fibrillation, stroke, transient ischemic attack, systolic dysfunction, diastolic dysfunction, myocarditis, atrial tachycardia, ventricular fibrillation, endocarditis, arteriopathy, vasculitis, atherosclerotic plaque, vulnerable plaque, acute coronary syndrome, acute ischemic attack, sudden cardiac death, peripheral vascular disease, coronary artery disease (CAD), peripheral artery disease (PAD), and cerebrovascular disease. In some embodiments, cardiovascular disease can be treated or prevented by administration of the disclosed probiotic composition. In some embodiments, cardiovascular disease can be treated or prevented by fecal microbiota transfer (FMT) from subjects whose microbiome includes Emergencia timonensis.

In some embodiments, the method can comprise administering an effective amount of a probiotic composition to the gastrointestinal tract or the urogenital tract of the subject in need thereof. In some embodiments, the probiotic composition can comprise Emergencia timonensis and a pharmaceutically acceptable carrier or excipient. In some embodiments, the Emergencia timonensis can be selected from a strain that is 99% identical to strain SN18. In some embodiments, the Emergencia timonensis can be selected from a strain that is 99% identical to strain AM07-24. In some embodiments, the Emergencia timonensis can be selected from a strain that is 99% identical to isolate MGYG-HGUT-01547. In still other embodiments, the Emergencia timonensis can be strain SN18. In some embodiments, the Emergencia timonensis can comprise a combination of at least two of strain SN18, strain AM07-24, isolate MGYG-HGUT-01547, and combinations thereof. In some embodiments, the Emergencia timonensis can express a protein having an amino acid sequence that is at least 90% identical to the amino acid sequence of SEQ ID NO:1 and/or the amino acid sequence of SEQ ID NO:2.

In some embodiments, the probiotic composition can be formulated for a human or an animal. In some embodiments, the probiotic formulation can be compounded as a food or as a pharmaceutical preparation. In still other embodiments, the pharmaceutical preparation can be a capsule, a suppository, an enema, a powder, a foam, a gel, a suspension or a fecal transplant. In some embodiments, the pharmaceutical preparation can be a vaginal suppository. In some embodiments, the pharmaceutical preparation can be formulated to be released in the intestine of a subject following administration of the probiotic composition to the subject. In some embodiments, the pharmaceutical preparation can be formulated to be released in the urogenital tract of a subject following administration of the probiotic composition to the subject. In some embodiments, the probiotic composition can comprise from 1 to 100 billion, from 1 to 10 billion, from 1 to 5 billion, or from 10 million to 1 billion colony forming units. In some embodiments, the probiotic composition comprises sufficient number of Emergencia timonensis to establish a colony of Emergencia timonensis in the cavity to which it has been delivered.

In some embodiments, the effective amount of the probiotic composition can be an amount that reduces TMA or TMAO at or below a level of TMA or TMAO found in a healthy subject population, i.e., population not suffering from the disease or condition treatable by reduction of the amount of TMA or TMAO. In some embodiments, the range of urinary TMA levels in healthy subjects are ranges between 0.70 μM and 4.39 μM. In some embodiments, reducing the amount of TMA by the methods of the disclosure results in a reduction in the amount of TMAO in the patient.

In some embodiments, the subject can have or can be suspected of having cardiovascular disease, thrombosis, trimethylaminuria, bacterial vaginosis, or a combination thereof. In some embodiments, the subject can have an elevated TMA level or an elevated TMAO level. In some embodiments, the TMA level and the TMAO level can be determined by measuring the elevated TMA level and the elevated TMAO level in the urine using liquid chromatography-mass spectrometry (LC/MS/MS).

Unless specifically defined herein, all terms used herein have the same meaning as they would to one skilled in the art of the present invention. The use of the term “or” in the claims is used to mean “and/or” unless explicitly indicated to refer to alternatives only or the alternatives are mutually exclusive, although the disclosure supports a definition that refers to only alternatives and “and/or.” The words “a” and “an,” when used in conjunction with the word “comprising” in the claims or specification, denote one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element. Additionally, the words “herein,” “above,” and “below,” and words of similar import, when used in this application, shall refer to this application as a whole and not to any particular portions of the application.

Disclosed are materials, compositions, and components that can be used for, can be used in conjunction with, can be used in preparation for, or are products of the disclosed methods and compositions. It is understood that, when combinations, subsets, interactions, groups, and the like, of these materials are disclosed, each of various individual and collective combinations is specifically contemplated, even though specific reference to each and every single combination and permutation of these components and the like, may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

All publications cited herein and the subject matter for which they are cited are hereby specifically incorporated by reference in their entireties.

The following examples are provided to illustrate certain particular features and/or embodiments of the disclosure. The examples should not be construed to limit the disclosure to the particular features or embodiments described.

### EXAMPLES

### Example 1. Discovery of Proteins

Select genomes were identified that have the pyrrolysine machinery. Among these was Emergencia timonensis. Genomic data sets were accessed on Jan. 15, 2020 from publicly available sequencing projects. The Emergencia timonensis genomes were analyzed using the alternate translation table in which the codon for stop is reassigned as pyrrolysine.

Predicted proteins in each genome were initially identified using Prodigal v.2.6.3 using single genome mode and functional annotation was determined using Hmmer v.3.1, with the hmmscan option (1E-10 cutoff), with top hits only, against the Pfam database v.31 and Tigrfam database v.15.0.

For Emergencia timonensis genomes, in which enzymes were identified for the synthesis of pyrrolysine, an alternate protein prediction procedure was applied that reassigned TAG codons from stop to readthrough. To accomplish this task, the source code for Prodigal was modified by adding a custom translation table that has TAG readthrough and retains all three canonical bacterial start codons. Proteins with in-frame stop codons in the relevant genes were then manually inspected to determine if the region containing and following the stop codon was conserved in comparisons to homologues containing pyrrolysine at a similar position.

### Example 2. Isolating and Culturing Emergencia timonensis

Exemplary strains of Emergencia timonensis are available for purchase from DSMZ. Medium 104 was used for culture, and prepared as follows, as described on the DSMZ website.

PYG MEDIUM (modified) Trypticase peptone 5.00 g, Peptone 5.00 g, Yeast extract 10.00 g, Beef extract 5.00 g, Glucose 5.00 g, K₂HPO₄ 2.00 g, Tween 80 1.00 mL, Salt solution (see below) 40.00 mL, Resazurin 1.00 mg, Vitamin K1 solution (see below) 0.20 mL, Haemin solution (see below) 10.00 mL, Cysteine-HCl x H2O 0.50 g, Distilled water 950.00 mL. The vitamin K1, haemin solution and the cysteine were added after the medium was boiled and cooled under CO₂. pH was adjusted to 7.2 using 8 N NaOH, distributed under N2 and autoclaved.

Salt solution: CaCl₂)×2H2O 0.25 g, MgSO₄×7 H₂O 0.50 g, K₂HPO₄ 1.00 g, KH₂PO₄ 1.00 g, NaHCO3 10.00 g, NaCl 2.00 g, Distilled water 1000.00 mL.

Haemin solution: 50 mg haemin was dissolved in 1 mL 1 N NaOH; and made up to 100 mL with distilled water. The solution was store refrigerated. Vitamin K1 solution: 0.1 mL of vitamin K1 was dissolved in 20 mL 95% ethanol and filter sterilized. The solution was store refrigerated in a brown bottle.

### Example 3. Genomic Analysis and Implementation of a Custom Protein Translation Table

Predicted proteins in the Emergencia timonensis SN18 genome were initially identified using Prodigal v.2.6.3 using single genome mode, and functional annotation was determined using hmmer v.3.1 with the hmmscan option (1E-15 cutoff) against the Tigrfam database v.15.0. The genes required for the synthesis of pyrrolysine were identified so an alternate protein prediction procedure was applied to reassign TAG codons from stop to readthrough, resulting in the prediction of the pyrrolysine containing trimethylamine and dimethylamine methyltransferase proteins. Further investigation to determine if these proteins were expressed was performed via cultivation and proteomics as described below.

### Cultivation and Proteomics

E. timonensis SN18 culturing was performed in Hungate or Balch tubes (Chemglass Life Sciences) at 37° C. and set up in an anaerobic chamber (Anaerobe Systems) under an atmosphere of 4% H2, 4% CO2, and N2 as the balance. Standard cultures were grown in brain heart infusion (BHI) medium for 72 h.

BHI medium formulation is as follows: Brain Heart Infusion 17.50 g, Proteos Peptone 10.00 g, Dextrose 2.00 g, Sodium Chloride 5.00 g, Disodium Phosphate 2.50 g, Yeast Extract 5.00 g, Hemin (0.1% solution) 5.00 mL, Vitamin K1 (1.0% solution) 0.20 mL,

Resazurin (0.025% solution) 4.00 mL, L-Cysteine Hydrochloride (25.0% solution) 2.00 mL, and DI Water 1.00 L. Final pH: 7.3±0.3 at 25° C. Final volume: 5.0 mL±0.5 mL.

Prior to proteomic extraction, cells were centrifuged at 1,500×g for 10 min and collected. The pellet was rinsed and resuspended in PBS buffer for whole protein extraction. Dithiothreitol was added to a final concentration of 5 mM and incubated for 1 hr at 56° C. Then iodoacetamide was added to a final concentration of 10 mM and incubated for 1 hr at room temperature. Then 1 μg of trypsin was added and allowed to incubate overnight at 37° C. After trypsin digestion formic acid was added to a final concentration of 0.6% and incubated for 1 hr at 37° C. Then samples were centrifuged for 5 min at 10,000×g to precipitate insoluble particles. The samples on the cartridge were then washed twice with 5% methanol. The eluate was sequenced at the OSU Mass Spectrometry Center (Corvallis, OR) using the Orbitrap Fusion Lumos mass spectrometer (Thermo Fisher Scientific). Raw files were processed by MaxQuant v.2.0.1.0. A custom database was generated for Emergencia timonensis for searches using MaxQuant.

### Results

The predicted sequences for trimethylamine methyltransferase and dimethylamine methyltransferase are shown below in Table 1 and Table 2 respectively. The proteomic sequence that was confirmed with our experiment is bold and underlined; in the table below, each protein shows corresponding data for these residues.

TABLE 1
FLKM01000008.1_1775
Trimethylamine methyltransferase

| | TAQSPVGAPEHAMCSAAV |
|---|---|
| Peptide | GQLGHYYGIPTNVGGTQADSK |
| sequence | SEQ ID NO: 4 |
| Intensity | 6813700 |
| Length | 39 |
| Mass | 4035.9207 |
| MS/MS Count | 1 |
| Score | 30.996 |

TABLE 2
FLKM01000008.1_1776
dimethylamine methyltransferase

| | ATMPLFYGAMPNLGFY |
|---|---|
| Observed Peptide | TKPDGPVDNWAMLLPEGK |
| sequence | SEQ ID NO: 5 |
| Intensity | 6646800 |
| Length | 34 |
| Mass | 3740.8081 |
| MS/MS Count | 2 |
| Score | 13.641 |

TABLE 3
amino acid sequences

| SEQ | | |
|---|---|---|
| ID NO | Protein | Amino Acid Sequence |
| 1 | Trimethylamine | MGKFTHETDITGYNILSNEDLDALHESTLQLLEDYGLQMHGKEALDILAGAGCEVD |
| | methyltransferase | YENDRVKFPRNLVNDAIEKCPAEFTLCGRDPAKDTVVGGKKVAYKNFGTGVFIFDP |
| | | YTGELRESTKEDLGNVARFIDAIPEIDVFSIAVTAGDVNQKVRSLHEAEVVENNLT |
| | | KNFAHDLEGVKNTQRFIDMAAAIQGGYDKLRERPIVAMGACPNSPLEINENETSII |
| | | ILSARHGLPIDILSMGLCGATTPATIAGTLVCTNAEILGGIVLAQIVNPGNKILYG |
| | | SSTTIMDMKTAQSPVGAPEHAMCSAAVGQLGHYYGIPTNVGGTQADSKCFDVQLGH |
| | | EKTMTNMLPALTGSNVIYGMGMMEMGMTMSYEQLLTDAEIVRMTRRILQGIPVNPD |
| | | TMALDVIKAVGPANNYLAQKHTRKYMRQELSTTQYIDRTQRETWEKKGGMTMTEAT |
| | | KAKAIDILENYEVTPLPDDVKKRIHDIVVEGEEEAEELARFQAKKK |
| 2 | Trimethylamine | MSFQFSKETRGYRLLTDQGIVTIHNATMDLMGQYGVRIFGTEAKKIYEEAGCDVDW |
| | methyltransferase | ESNMVKFPRELVEWAIEVTPGTFTMCGRHPKDDFVLGDGTVSFTSFGTGLQMFDLE |
| | | TGEVRDTDTEDLENFARFCDAIPEINAFTTAVAAQDAPGPLKDFYEAEAIFKNTTK |
| | | HAILDAENGHNAKAIIDMGIAIAGSLQKLQERPFFTLCMCPNSPLEIHDGASQVII |
| | | ETAKAGLPISILSMGLAGGTTPATLTGTFVVTNAEILAGIVLTQLIHEGNPVIYGS |
| | | STTVMDMRKATSPVGAPEHGMFGAMVAQMGKYYDIVTKVGGTQADSKVMDVQIGHE |
| | | KTLTGLMPVLAGVDLVYGLGMIDMGMAISYEQFLMDAEFVRMFKRTEKEVMVDEDA |
| | | LALSVIKAVGPAGNYLSQRHTLAHMRDEISTTALIDRQMREKWQKDGAKDMPQRAR |
| | | ELAKEILAKHRPEPLPADVQAELAAIIKREGDLLCR |
| 3 | dimethylamine | MGDQKKFFTRMGDGSPVYMTEEEIRADIKAGIDDAVLRGKIDPLSEEDTERIYEIV |
| | methyltransferase | TMPGNIVGVQPGMEIVTSNDSGSDKISTKCGISTSRDTNALIHERVLGADSVDIGY |
| | | SDYNYKTVKGVAKREATVLKQALTKATMPLFYGAMPNLGFYTKPDGPVDNWAMLLP |
| | | EGKIKEAMAAQEEAVDHAVRDIVYVAEQMNDVGADAFMLDTSGAAGDADLLVSLKA |
| | | CEIIREKFPDMPVEIGMAGEFVLGMHGKLKYNGTRLAGLYPHKQVKVVEEAGASIF |
| | | GAVVNTNCNKSFPWNIARVCTFIKACTDVAEIPVHANAGMGVCGIPMVENLPSGDP |
| | | DVVSRADKCLIEIGKVDGLQIGVLGAEATHALAAGMGGIRTAGDLVMRLQLAKKMK |
| | | INEAKAYVAEKLSVSVEELCDIVTMNDKREELGLGLAHVEPNAEANAGMEAKFRIQ |
| | | EVLDIKVNSVERFKERAGLK |

TABLE 4
16s RNA gene sequence

SEQ ID NO: 6
Strain SN18
Accession number: LN998061

| | |
|---|---|
| 1 | agagtttgat cctggctcag gatgaacgct ggcggcgtgc ctaacacatg caagtcgagc |
| 61 | gagaagccat tgactgaaac ttcggtagaa ggatatggtg gaaagcggcg gacgggtgag |
| 121 | taacgcgtag gcaacctgcc ccttacagag ggatagccat tggaaacgat gattaaaacc |
| 181 | tcataacgca tcccccycac atggagggga wgccaaagat tcatcggtaa gggatgggcc |
| 241 | tgcgtctgat tagcttgttg gcggggtaac ggcccaccaa ggcgacgatc agtagccgac |
| 301 | ctgagagggt gatcggccac attggaactg agacacggtc caaactccta cgggaggcag |
| 361 | cagtggggaa tattgcacaa tgggcgaaag cctgatgcag caacgccgcg tgagggatga |
| 421 | aggccttcgg gtcgtaaacc tctgtccttg gggaagaaac aaatgacggt acccatggag |
| 481 | gaagccccgg ctaactacgt gccagcagcc gcggtaatac gtagggggcg agcgttatcc |
| 541 | ggaattattg ggcgtaaaga gtgcgtaggt ggttacctaa gcgcagggtc taaggcaatg |
| 601 | gctcaaccat tgttcgccct gcgaactggg ctacttgagt gcaggagagg aaagcggaat |
| 661 | tcctagtgta gcggtgaaat gcgtagatat taggaggaac accagtggcg aaggcggctt |
| 721 | tctggactgt tactgacact gaggcacgaa agcgtgggga gcaaacagga ttagataccc |
| 781 | tggtagtcca cgccgtaaac gatgagcact aggtgtcggg gccgcaaggc ttcggtgccg |
| 841 | cagttaacgc attaagtgct ccgcctgggg agtacgcacg caagtgtgaa actcaaagga |
| 901 | attgacgggg acccgcacaa gcagcggagc atgtggttta attcgaagca acgcgaagaa |
| 961 | ccttaccagg acttgacate ccactgacag atccctaacc ggatccttct tcggacagtg |
| 1021 | gagacaggtg gtgcatggtt gtcgtcagct cgtgtcgtga gatgttgggt taagtcccgc |
| 1081 | aacgagcgca acccttgcca ttagttgcca tcattcagtt gggcactcta atgggactgc |
| 1141 | cggggacaac tcggaggaag gtggggatga cgtcaaatca tcatgcccct tatgttctgg |
| 1201 | gctacacacg tgctacaatg gccggtacag caagaagcaa aaccgcgagg tggagcaaat |
| 1261 | ctcaaaaacc ggtcccagtt cggactgcag gctgaaaccc gcctgcacga agccggagtt |
| 1321 | gctagtaatc gtggatcaga atgccacggt gaatgegttc ccgggtcttg tacacacegc |
| 1381 | ccgtcacacc atggaagttg ggggtgcccg aagccggcag ggaaatatgc tgtctaaggc |
| 1411 | aaaaccaatg actggggtga agtcgtaaca aggtagccgt |

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for reducing the amount of trimethylamine (TMA) in a subject, the method comprising administering an effective amount of a probiotic composition comprising Emergencia timonensis and a carrier or excipient to the subject in need thereof

2. The method of claim 1, wherein the subject in need thereof has or is suspected of having cardiovascular disease, thrombosis, trimethylaminuria, bacterial vaginosis, or a combination thereof.

3. The method of claim 2, wherein the subject in need thereof has an elevated TMA level and/or an elevated trimethylamine-N oxide (TMAO) level, wherein the elevated TMA level and/or the elevated TMAO level are quantified by measuring the elevated TMA level and/or the elevated TMAO level in urine using liquid chromatography-mass spectrometry.

4. The method of claim 3, wherein the effective amount of the probiotic composition comprises an amount that reduces the elevated TMA level and/or the elevated TMAO level at or below a level of TMA and/or a level of TMAO found in a healthy subject.

5. The method of claim 1, wherein the Emergencia timonensis expresses a protein having an amino acid sequence that is at least 90% identical to amino acid sequence SEQ ID NO:1 and/or at least 90% identical to amino acid sequence SEQ ID NO:2.

6. The method of claim 5, wherein the protein comprises a TMA utilizing protein, and wherein the Emergencia timonensis produces the TMA utilizing protein under anaerobic growth conditions.

7. The method of claim 1, wherein the probiotic formulation is compounded as a food or as a pharmaceutical preparation.

8. The method of claim 7, wherein the pharmaceutical preparation is a capsule, a suppository, an enema, a powder, a foam, a gel, a suspension or a fecal transplant.

9. The method of claim 8, wherein the pharmaceutical preparation is formulated to be released in the intestine or the urogenital tract of the subject following administration of the probiotic composition to the subject.

10. The method of claim 1, wherein the probiotic composition comprises from 1 colony forming unit to 100 billion colony forming units, from 1 colony forming unit to billion colony forming units, from 1 colony forming unit to 5 billion colony forming units, or from 10 million colony forming units to 1 billion colony forming units.

11. A method for treating or preventing a condition treatable by providing a bacterium capable of producing a trimethylamine (TMA) utilizing protein or reducing and/or maintaining the level of TMA in a gastrointestinal tract or a urogenital tract of a subject in need thereof, the method comprising administering an effective amount of a probiotic composition comprising Emergencia timonensis and a carrier or excipient to the subject in need thereof.

12. The method of claim 11, wherein the subject in need thereof has or is suspected of having cardiovascular disease, thrombosis, trimethylaminuria, bacterial vaginosis, or a combination thereof.

13. The method of claim 12, wherein the subject in need thereof has an elevated TMA level and/or an elevated trimethylamine-N oxide (TMAO) level, wherein the elevated TMA level and/or the elevated TMAO level are quantified by measuring the elevated TMA level and/or the elevated TMAO level in urine using liquid chromatography-mass spectrometry.

14. The method of claim 11, wherein the effective amount of the probiotic composition comprises an amount that reduces the elevated TMA level and/or the elevated TMAO level at or below a level of TMA and/or a level of TMAO found in a healthy subject.

15. The method of claim 11, wherein the Emergencia timonensis expresses a protein having an amino acid sequence that is at least 90% identical to amino acid sequence SEQ ID NO:1 and/or at least 90% identical to amino acid sequence SEQ ID NO:2.

16. The method of claim 15, wherein the protein comprises a TMA utilizing protein, and wherein the Emergencia timonensis produces the TMA utilizing protein under anaerobic growth conditions

17. The method of claim 11, wherein the probiotic formulation is compounded as a food or as a pharmaceutical preparation.

18. The method of claim 17, wherein the pharmaceutical preparation is a capsule, a suppository, an enema, a powder, a foam, a gel, a suspension or a fecal transplant.

19. The method of claim 18, wherein the pharmaceutical preparation is formulated to be released in the intestine or the urogenital tract of the subject in need thereof following administration of the probiotic composition to the subject in need thereof

20. The method of claim 11, wherein the probiotic composition comprises from 1 colony forming unit to 100 billion colony forming units, from 1 colony forming unit to billion colony forming units, from 1 colony forming unit to 5 billion colony forming units, or from 10 million colony forming units to 1 billion colony forming units.
